(19) United States
(12) Patent Application Publication
(10) Pub. No.: US 2018/0011081 A1
(43) Pub. Date: Jan. 11, 2018

(54) METHOD FOR DETECTING HELICOBACTER PYLORI

(71) Applicant: Kibion GmbH, Bremen (DE)

(72) Inventors: Grischa Wagner, Bremen (DE); Marko Silvestric-Scheel, Bremen (DE)

(73) Assignee: Kibion GmbH, Bremen (DE)

(21) Appl. No.: 15/544,199

(22) PCT Filed: Jan. 12, 2016
(86) PCT No.: PCT/EP2016/000035

§ 371 Date: Jul. 17, 2017

(30) Foreign Application Priority Data
Jan. 22, 2015 (DE) 10 2015 000 626.6

(51) Int. Cl. G01N 33/497; G01N 21/3504; G01N 21/61

(52) U.S. Cl. CPC G01N 33/497; G01N 21/61; G01N 21/3504; G01N 2201/0233; G01N 2033/4977

(57) ABSTRACT

A method through which a more rapid detection of Helicobacter pylori in a gaseous sample is practicable, in which the ¹³C content is measured only until a minimum number of measurement values of the ¹³C content meets a standard deviation to be specified. The known ¹³C urea breath test has become established for clinical diagnosis for detecting Helicobacter pylori infections and known methods for detecting Helicobacter pylori provide that each method step corresponds to a fixed, specified time, which is disadvantageous, especially for performing a large number of such tests.

### STATEMENT OF RELATED APPLICATIONS

The application is the US PCT National Phase of International Application No. PCT/EP2016/000035 having an International Filing Date of 12 Jan. 2016, which claims priority on German Patent Application No. 10 2015 000 626.6 having a filing date of 22 Jan. 2015.

### BACKGROUND OF THE INVENTION

### Technical Field

The invention relates to a method for detecting Helicobacter pylori by means of non-dispersive infrared spectroscopy with use of ¹³C-labeled urea, wherein firstly a measurement chamber is flushed with CO₂-free gas, a gaseous sample is admitted into the measurement chamber, the sample distributes itself homogeneously and the ¹³C content in the sample is measured.

### Prior Art

The ¹³C urea breath test has become established for clinical diagnosis for detecting Helicobacter pylori infections, which are often the cause of diseases of the gastrointestinal tract. In this breath test, urea labeled with ¹³C is administered to the person affected. Helicobacter pylori produces an enzyme named urease, which degrades the urea inter alia to CO₂. In the process, the ¹³C carbon isotope of the labeled urea is incorporated into the CO₂ molecule. Thus, for detecting an infection with this bacterium the ratio between ¹³CO₂ and ¹²CO₂ has to be determined. For this, a suitably labeled substrate is administered to the person affected and the breath analyzed after a certain time. For the analysis of the breath, non-dispersive infrared spectroscopy (NDIR spectroscopy) is used. For this, filtered infrared radiation passes through a measurement chamber which contains the gas to be analyzed. By measurement of the absorption spectrum by means of an infrared detector, statements can be made about the ¹³C/¹²C content in the gas. With an elevated content of ¹³C it can be assumed that the person is infected with the bacterium Helicobacter pylori.

Known methods for detecting Helicobacter pylori provide that firstly a measurement chamber in which the detection is to be performed is flushed with a CO₂-free gas. This flushing takes place according to a fixed time, which is usually 30 secs to 60 secs. After the flushing of the measurement chamber, the gaseous breath sample with the in some cases elevated content of ¹³CO₂ is admitted. The admission of the sample into the measurement chamber is effected after a specified time of 20 secs to 40 secs. After this time, the inlet valve of the measurement chamber is closed and the sample distributes itself homogenously in the measurement chamber. This homogenization is especially important in order to be able reliably to determine the ¹³C ratio and the ¹²C ratio. In practice, the waiting time is about 40 secs to 60 secs. After this time has elapsed, the actual measurement on the gaseous sample takes place. As a rule the measurement lasts 15 secs to 30 secs, depending on how many measurement values are to be taken.

Particularly in the clinical field, a large number of the tests described above concerning a possible infection with Helicobacter pylori must be performed daily. Since the method described is very time-intensive, it proves unsuitable for the management of a large number of tests.

### BRIEF SUMMARY OF THE INVENTION

The invention is therefore based on the problem of creating a method by which a more rapid detection of Helicobacter pylori in a gaseous sample is practicable.

One method for solving this problem is a method for detecting Helicobacter pylori by means of non-dispersive infrared spectroscopy with use of ¹³C-labeled urea, wherein firstly a measurement chamber is flushed with CO₂-free gas, a gaseous sample is admitted into the measurement chamber, the sample distributes itself homogeneously and the ¹³C content in the sample is measured, characterized in that the measurement of the ¹³C content is only carried out until a minimum number of measurement values of the ¹³C content meets a standard deviation to be specified. According to this, it is provided according to the invention that the measurement of the ¹³C content is carried out only until a minimum number of measured values of the ¹³C content meets a standard deviation to be specified. This in particular represents a time advantage. Often after a short time the sample in the measurement chamber is already homogenized such that the recording of measurement values can begin. Hence considerable time can be saved through this method, since it is no longer necessary as always before to wait for a certain time until the recording of the measurement values can be effected. Rather, according to the invention waiting for the recording of the measurement values is only for exactly as long as necessary. The standard deviation, in particular the standard deviation to a mean value, is then specified in advance or determined on the basis of the measurement values.

Preferably, the present invention can provide that the ¹³C content in the sample is already measured during the homogenization of the sample in the measurement chamber. Through the continuous or cyclical measurement of the ¹³C content during the homogenization or during the whole method, it becomes possible in particular to monitor the homogenization of the sample in the measurement chamber. Should measurement values already appear during the homogenization which differ only slightly from the target value, these values can already be used as result values. Here it is provided according to the invention that the last measurement values are always stored for the case that these values already meet the requirements for assessable measurement values.

In particular, the invention provides that the first recorded measurement values of the ¹³C content in the sample, in particular the first ten, preferably the first twenty measurement values which meet the standard deviation are used as measurement results. Here the number of measurement values necessary depends on the specified precision or reliability. Here also, by reduction of the measurement values necessary, the time which is taken up by the method can be reduced.

A further practical example of the invention provides that as soon as the ¹³C measurement values meet the standard deviation of at most 1‰, preferably at most 0.2‰, the ¹³C measurement values are assessed as measurement results. Here the values for the standard deviation are variable and can be altered depending on the application and requirement. Thus it is possible that the standard deviation is also specified as 10‰ or 5‰ or even 0.1‰.

Further, the invention can provide that the flushing of the measurement chamber and the admission of the sample into the measurement chamber is only performed until a threshold value, to be specified, of the CO₂ content or the ¹²C content in the measurement chamber is reached. For this, the CO₂ content or the ¹²C content in the measurement chamber is measured during the flushing of the measurement chamber and during the admission of the sample into the measurement chamber. Initially, any gas is present in the measurement chamber. By flushing of the measurement chamber with a CO₂-free gas and simultaneous pumping out of the chamber with a pump, the removal of any residual gas, in particular carbon or CO₂, from the chamber is effected. During this flushing procedure, the gas is continuously or cyclically examined spectroscopically. As soon as the CO₂ content in the measurement chamber is 0.1% or 0.05%, the flushing is ended. These values can be altered as required.

In the next step, the sample or the breath sample of the person affected is then passed into the measurement chamber. Also during this admission of the breath sample into the measurement chamber, a pump can be operated in order to convey the breath sample into the measurement chamber. During the admission of the sample, the measurement of the CO₂ content or the CO₂ volume content continues. As soon as this content is at least 0.2% or 0.5%, the inlet valve is closed so that no more gas can flow into the chamber. This volume content has been found sufficient for the determination of ¹³C.

The present invention further provides that the ¹³C content and the CO₂ content or ¹²C content are determined by sensors which are read off by a control unit and the control unit compares the measurement values of the sensors with the threshold values to be specified. Apart from this, the threshold value or the standard deviation can be preset via this control unit. As soon as a breath sample is passed into the measurement chamber, the procedure described above takes place automatically. However, it can also be provided that the individual steps, depending on the result, have to be initiated individually. Further, the invention can provide that the control unit controls several measurement procedures running in parallel or coordinates several samples for the measurement of the ¹³C content.

A further particularly advantageous practical example of the present invention provides that pumps and/or valves for regulating a gas flow into or out of the measurement chamber are regulated by the control unit. Thus valves are closed or pumps switched on or off and/or measurement values read off depending on the measurement values.

### BRIEF DESCRIPTION OF THE DRAWING

A preferred practical example of the method according to the invention is explained in more detail on the basis of a diagram below.

FIG. 1 is a graph of the variation of carbon content in a measurement chamber with time.

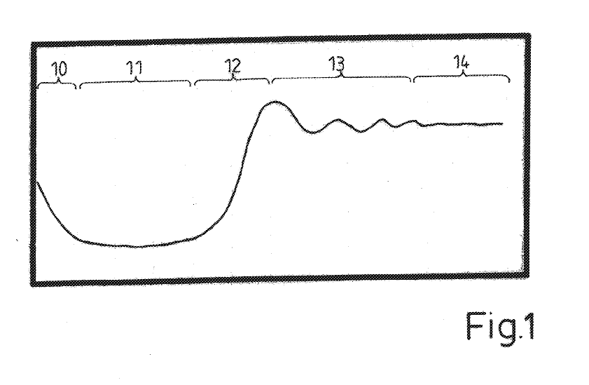

### DETAILED DESCRIPTION OF PREFERRED EMBODIMENTS

In the diagram, the variation of the carbon content, in particular the ¹³C content, in the measurement chamber with time during the method according to the invention is plotted. The y-axis represents in arbitrary units the relative content of the carbon in the measurement chamber.

In a first phase 10 of the method, the measurement chamber is filled with any gas and thus with any content of carbon or CO₂. For example, a measurement for the detection of Helicobacter pylori had just been finished. In the second phase 11, the measurement chamber is flushed, i.e. prepared for the actual measurement method. For this a CO₂-free gas is passed into the chamber and at the same time a pump which is connected to the chamber is switched on. Through the flushing and pumping, the carbon content in the measurement chamber decreases rapidly. Already during this second phase 11 of the method, the carbon content is determined by NDIR spectroscopy. As soon as the carbon content has gone below a defined threshold value, the flushing is ended by ending the admission of the CO₂-free gas.

In the third phase 12 of the method, the breath sample of the person affected is passed into the measurement chamber. Since the CO₂ content in the breath sample is high, the carbon content climbs rapidly in the third phase 12. In this phase also, the carbon content is determined by spectroscopy. As soon as the CO₂, in particular the ¹²C and/or ¹³C, content meets a certain threshold value, the admission of the breath sample is also stopped by a valve being closed.

In the following fourth phase 13, a homogenization of the CO₂-containing breath sample in the measurement chamber takes place. During the fourth phase 13, a specific measurement of the ¹³C content (fifth phase 14) already takes place. Thus the fourth phase 13 passes smoothly into the fifth phase 14. If the ¹³C measurement values already reveal that the deviations meet a preset standard deviation, these measurement values are already assessed as measurement results. Usually 10 to 20 such measurement results or points are recorded in order to achieve a reliable result concerning infection with Helicobacter pylori. After the fifth phase 14, the method can be continued according to the first phase 10.

### LIST OF SYMBOLS

10 1^{st} phase
11 2^{nd} phase
12 3^{rd} phase
13 4^{th} phase
14 5^{th} phase

What is claimed is:

1. A method for detecting Helicobacter pylori by means of non-dispersive infrared spectroscopy with use of ¹³C-labeled urea, comprising:
firstly a measurement chamber is flushed with CO₂-free gas;
a gaseous sample is admitted into the measurement chamber;
the sample distributes itself homogeneously and the ¹³C content in the sample is measured; and
the measurement of the ¹³C content is only carried out until a minimum number of measurement values of the ¹³C content meets a standard deviation to be specified.

2. The method as claimed in claim 1, wherein the ¹³C content in the sample is already measured during the homogenization of the sample in the measurement chamber.

3. The method as claimed in claim 1, wherein the first recorded measurement values of the ¹³C content in the sample which meet the standard deviation are used as measurement results.

4. The method as claimed in claim 1, wherein as soon as the ¹³C measurement values meet the standard deviation of at most 1‰, the ¹³C measurement values are assessed as measurement results.

5. The method as claimed in claim 1, wherein the flushing of the measurement chamber and the admission of the sample into the measurement chamber is performed only until a threshold value of the CO₂ content or the ¹²C content in the measurement chamber, to be specified, is reached.

6. The method as claimed in claim 1, wherein the CO₂ content or the ¹²C content in the measurement chamber is measured during the flushing of the measurement chamber and during the admission of the sample into the measurement chamber.

7. The method as claimed in claim 1, wherein the measurement chamber is flushed with the CO₂-free gas until the CO₂ content in the measurement chamber is at most 0.1% and thereupon the sample is admitted into the measurement chamber.

8. The method as claimed in claim 1, wherein the sample is admitted into the measurement chamber until the CO₂ volume content is at least 0.2%.

9. The method as claimed in claim 1, wherein the ¹³C content and the CO₂ content or ¹²C content are determined by sensors which are read off by a control unit and the control unit compares the measurement values of the sensors with the threshold values to be specified.

10. The method as claimed in claim 1, wherein pumps and/or valves for regulating a gas flow respectively into and out of the measurement chamber are regulated by the control unit.

11. The method as claimed in claim 1, wherein the first 10 recorded measurement values of the ¹³C content in the sample which meet the standard deviation are used as measurement results.

12. The method as claimed in claim 1, wherein the first 20 recorded measurement values of the ¹³C content in the sample which meet the standard deviation are used as measurement results.

13. The method as claimed in claim 1, wherein as soon as the ¹³C measurement values meet the standard deviation of at most 0.2‰, the ¹³C measurement values are assessed as measurement results.

14. The method as claimed in claim 1, wherein the measurement chamber is flushed with the CO₂-free gas until the CO₂ content in the measurement chamber is at most 0.05% and thereupon the sample is admitted into the measurement chamber.

15. The method as claimed in claim 1, wherein the sample is admitted into the measurement chamber until the CO₂ volume content is at least 0.5%.
